(19) United States
(12) Patent Application Publication
(10) Pub. No.: US 2024/0105285 A1
(43) Pub. Date: Mar. 28, 2024

(54) REACTION SYNTHESIS PATHFINDER

(71) Applicants: Merck Sharp & Dohme LLC, Rahway, NJ (US); MSD Czech Republic s.r.o., Prague 6 (CZ)

(72) Inventors: Pavel Vácha, Prague (CZ); Richard Dobis, Prague (CZ); Ondrej Micka, Veselí nad Luznicí (CZ); Nikita Vostrosablin, Prague (CZ); Martin Spale, Druzec (CZ); Josef Navrátil, Tismice (CZ); Jaroslav Cerman, Martinice v Krkonosích (CZ); Brian Lahue, Cambridge, MA (US); Thomas James Struble, Cambridge, MA (US); Zhengwei Peng, San Diego, CA (US); John Steele Debenham, Scotch Plains, NJ (US); Danny Asher Bitton, Prague (CZ); Petr Mejzlik, Prague (CZ)

(21) Appl. No.: 17/954,302

(22) Filed: Sep. 27, 2022

(51) Int. Cl. G16C 20/10

(52) U.S. Cl. CPC G16C 20/10

(57) ABSTRACT

A reaction pathfinder system can leverage historical chemical reaction data and determine a synthesis route in a reaction network. The reaction pathfinder system can account for various performance criteria of chemical reactions such as a highest yield for a molecule or a minimal number of reaction steps. A reaction pathfinder system receives a user request for a synthesis route including one of a user-specified molecule or a user-specified reaction. The reaction pathfinder system may then query a reaction network that has various synthesis routes and represents reactions of reactants to produce respective molecules. The reaction network may be composed of molecule nodes and reaction nodes. The reaction pathfinder system determines, using the reaction network, the synthesis route from the synthesis routes to fulfill the user's request.

### TECHNICAL FIELD

The subject matter described relates generally to synthesizing molecules and, in particular, to determining a synthesis route using a reaction network.

### BACKGROUND

As the cost of discovering new treatments rises, the demand increases for greater speed and efficiency in the drug discovery process. This process typically begins with a target clinical candidate and various hits and leads in the hopes of producing a successful drug. In early discovery, chemists who are responsible for discovering new molecules often need to perform chemical reactions on thousands of candidate molecules to identify ones that have the greatest potential for becoming drug candidates. After this discovery process has completed, chemists search for the fastest, most efficient, and safest routes to synthesize already-discovered molecules. Chemists often work across several different internal and external resources documenting historical reactions and experiments, exploring a vast number of possible reactions.

In recent decades, a vast amount of chemical reaction data has been accumulated. However, conventional systems allow viewing of only disconnected, historical reactions, which makes the exploration of networks of reactions very time consuming. Furthermore, conventional systems may simply return an arbitrary set of reactions for synthesizing a particular molecule without additional optimization. To speed up the exploration process, chemists can currently ignore the amount of reactions that are manually examined, but this comes at a cost of overlooking a desirable set of reactions for synthesizing a desired molecule. The drug development process currently faces a slow exploration process that consequently slows the discovery of new treatments.

### SUMMARY

A reaction pathfinder system uses historical chemical reaction data to determine a synthesis route in a reaction network. The reaction pathfinder system can account for various performance criteria of chemical reactions such as a highest yield for a molecule or a minimal number of reaction steps, which conventional systems for molecule discovery that simply return any set of reactions that produce the molecule cannot. For example, because the reaction pathfinder system can identify a synthesis route that has a minimal number of reaction steps for synthesizing a molecule, the reaction pathfinder system enables users to quickly synthesize a particular molecule in a laboratory. The reaction pathfinder system can also account for other performance criteria such as highest yield, enabling users to preserve chemical resources for subsequent experiments and achieve efficient, larger scale production of compounds. Accordingly, the reaction pathfinder system enables users to speed up and conserve resources for drug candidate exploration that conventional discovery systems do not enable.

In one embodiment, a reaction pathfinder system receives a request for a synthesis route including a user-specified molecule or a user-specified reaction. The reaction pathfinder system may then query a reaction network that has various possible synthesis routes and represents reactions of reactants to produce respective molecules. The reaction network may include molecule nodes and reaction nodes. The reaction pathfinder system selects the synthesis route from the possible synthesis routes using the reaction network to fulfill the user's request.

In some embodiments, the reaction pathfinder system may identify a synthesis route that optimizes for one or more synthesis performance criteria such as a minimum number of reaction steps for producing a molecule, a maximum yield of the molecule, or a maximum robustness for producing the molecule. The reaction pathfinder system can identify a reaction cycle within the reaction network, where the reaction cycle is a portion of the reaction network caused by a molecule child node for a particular molecule serving as a predecessor node of a molecule parent node for the same molecule. The reaction pathfinder system can identify one or more molecule nodes within the reaction cycle that each have an unassigned cost, and assign the one or more molecule nodes within the reaction cycle with a predetermined cost.

The reaction pathfinder system may generate the reaction network using historical reaction data. The reaction pathfinder system can assign initial cost values to the reaction network based on sources of molecules represented in the reaction network. The reaction pathfinder system can assign a first value for a first set of molecule nodes representative of molecules sourced from a third-party and assign a second value for a second set of molecule nodes representative of molecules produced from one or more of the molecules sourced from the third-party, where the first value can be smaller than the second value.

The reaction pathfinder system can determine a reaction node cost for a reaction node connected to molecule child nodes having assigned costs. In one embodiment, the reaction pathfinder system can, in response to determining to optimize against a synthesis performance criterion of a minimum number of reaction steps for producing a molecule, assign an initial cost of zero to a subset of the molecule nodes, determine the reaction node cost based on costs of molecule child nodes of the reaction node, and determine a molecule node cost of a molecule node based on a minimum of costs of a plurality of reaction child nodes of the molecule node. In another embodiment, the reaction pathfinder system can, in response to determining to optimize against a synthesis performance criterion of a maximum yield of a molecule, assign an initial cost of one to a subset of the molecule nodes, determine the reaction node cost based on a cost of a molecule node corresponding to a limiting reactant associated with the reaction node, and determine a molecule node cost based on both yield indicators and a cost of reaction child nodes of the molecule node. In yet another embodiment, the reaction pathfinder system can, in response to determining to optimize against a synthesis performance criterion of a maximum robustness for producing a molecule, assign an initial cost to a subset of the molecule nodes, where the initial cost is larger than a maximum number of experiments (e.g., as recorded by the reaction pathfinder system) as using a particular molecule of the synthesis routes in the reaction network. The reaction pathfinder system can then determine the reaction node cost based on a number of experiments of a reaction represented by the reaction node and based on a minimum cost of molecule child nodes of the reaction node. The reaction pathfinder system may also determine a molecule node cost based on a maximum cost of reaction child nodes of the molecule node.

The reaction pathfinder system can synthesize a molecule based on the determined synthesis route. In some embodiments, the reaction pathfinder system can generate a graphical user interface (GUI) that includes one or more user input controls for receiving the request. The reaction pathfinder system can generate the GUI to include a user input control for specifying a frequency at which a reaction network is to be updated (e.g., weekly). To update a reaction network, the reaction pathfinder system can identify a portion of the reaction network based on a presence of a particular synthesis route within the portion of the reaction network, the particular synthesis route optimizing for a synthesis performance criterion. The reaction pathfinder system can then update the portion of the reaction network to include additional molecule data or additional reaction data.

### BRIEF DESCRIPTION OF DRAWINGS

The disclosed embodiments have advantages and features which will be more readily apparent from the detailed description, the appended claims, and the accompanying figures (or drawings). A brief introduction of the figures is below.

FIG. 1 is a block diagram of a system environment in which a reaction pathfinder system operates, according to one embodiment.

FIG. 2 is a diagram of various data sources providing input to the reaction pathfinder system, according to one embodiment.

FIG. 3 illustrates a reaction network used by the reaction pathfinder system of FIG. 1, according to one embodiment.

FIG. 4 illustrates a reaction network including a reaction cycle, according to one embodiment.

FIG. 5 illustrates a reaction network including a reaction overlap, according to one embodiment.

FIG. 6 shows a graphical user interface (GUI) for using the reaction pathfinder system, according to one embodiment

FIG. 7 is a flowchart illustrating a process for determining a synthesis route, according to one embodiment.

FIG. 8 is a block diagram illustrating components of an example machine able to read instructions from a machine-readable medium and execute them in a processor (or controller), according to one embodiment.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed. It is noted that wherever practicable similar or like reference numbers may be used in the figures and may indicate similar or like functionality. Similar or like elements may be referred to individually using the same reference numeral followed by a different letter, and collectively by the reference numeral alone. The figures depict embodiments of the disclosed system (or method) for purposes of illustration only.

### System Overview

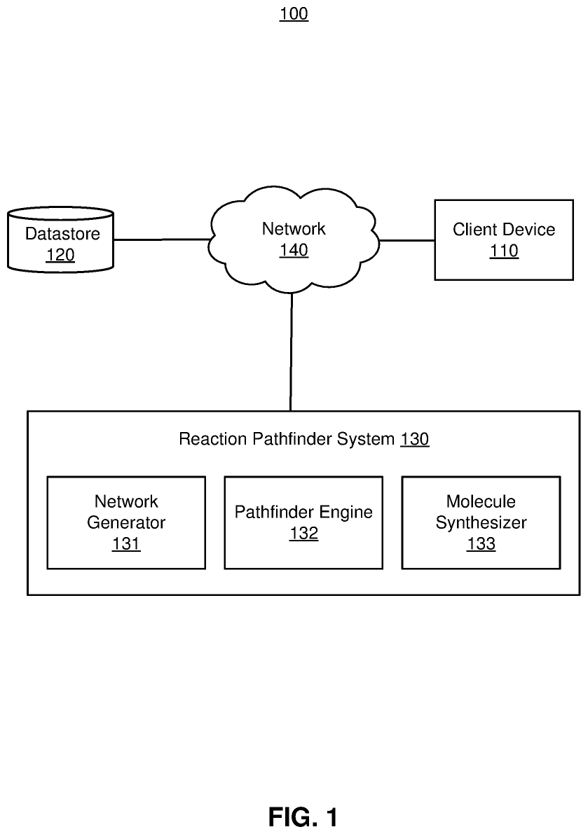

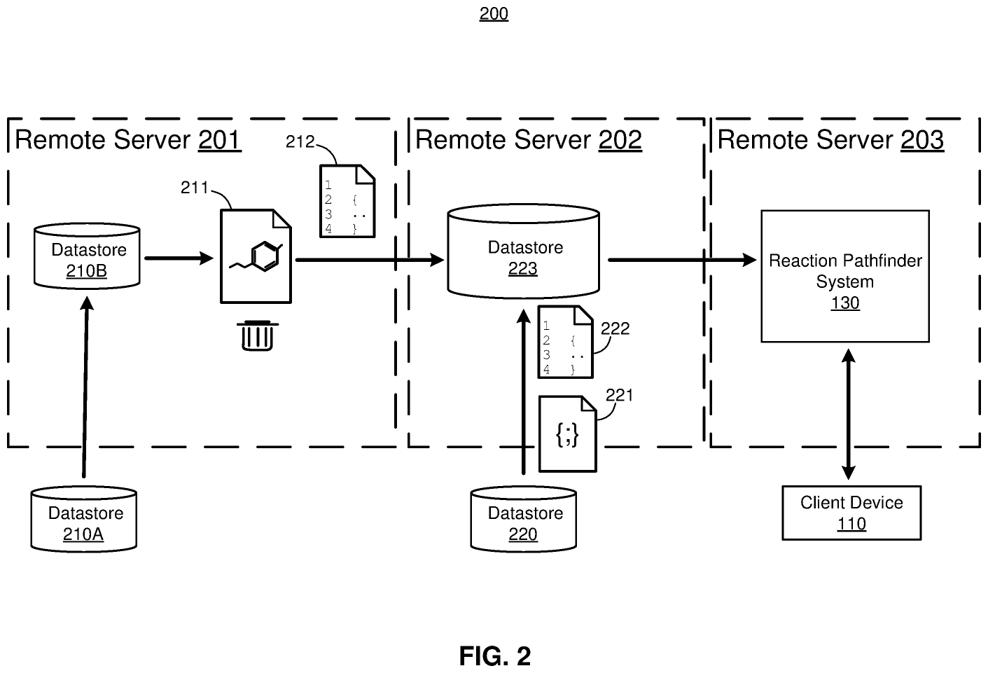

FIG. 1 is a block diagram of a system environment 100 in which a reaction pathfinder system 130 operates, according to one embodiment. The system environment 100 includes a client device 110, a datastore 120, a reaction pathfinder system 130, and a network 140. A user may submit a request to the reaction pathfinder system 130 via the client device 110 to determine a set of one or more chemical reaction steps, referred to as a “synthesis route,” for synthesizing a molecule. The terms “synthesize” and “react” may be used interchangeably to refer to the rearrangement of molecular structure of one or more substances (i.e., reactants) to be converted into one or more different substances (i.e., products). The user may request that the reaction pathfinder system 130 determine a synthesis route that optimizes for a performance metric of synthesizing the molecule (e.g., yielding the highest amount of a desired molecule). The system environment 100 may have alternative configurations than shown in FIG. 1, including for example, different, fewer, or additional components. For example, the system environment 100 may include additional client devices or datastore to which the reaction pathfinder system 130 is coupled via the network 140. FIG. 2 shows various data sources that may provide input to the reaction pathfinder system 130.

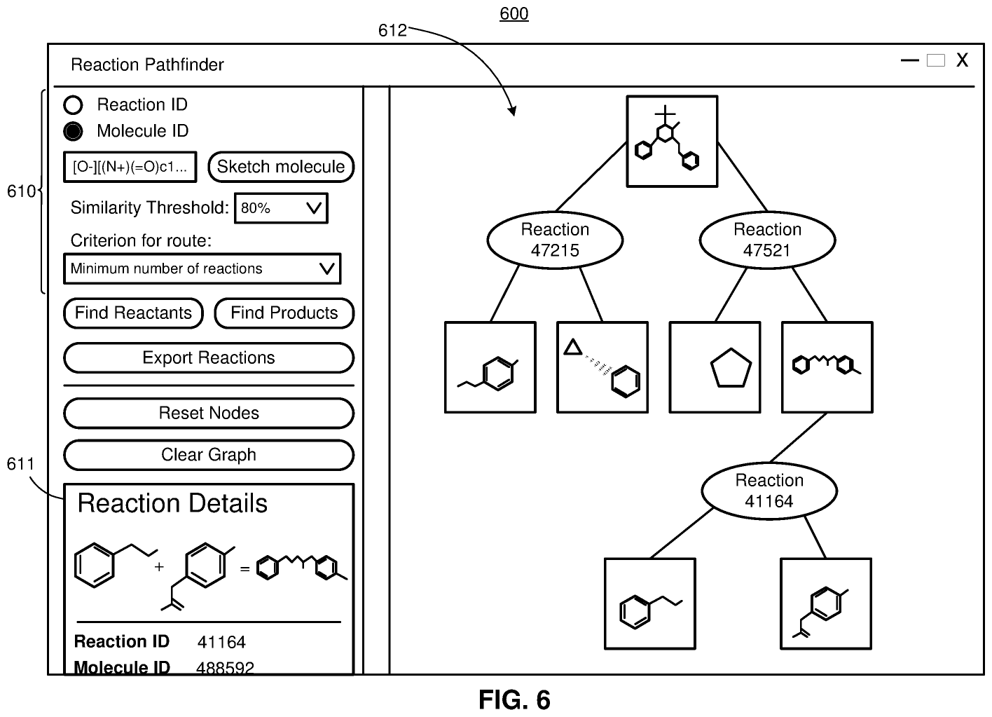

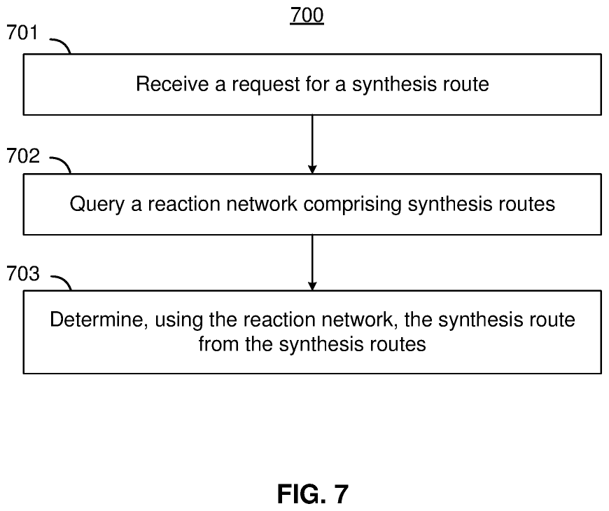

The client device 110 is an example of a computing device for users to request a synthesis route as enabled by the reaction pathfinder system 130. For example, the reaction pathfinder system 130 may provide for display at the client device 110 an interface to specify parameters of a requested molecule, a criterion for a synthesis route, or a combination thereof (e.g., as depicted in FIG. 6). In some embodiments, the client device 110 is a computer system, such as a desktop or a laptop computer. Alternatively, the client device 110 may be a device having computer functionality, such as a mobile telephone, a smartphone, or another suitable device. The client device 110 is configured to communicate with the reaction pathfinder system 130 via the network 140 (e.g., using a native application executed by the client device 110 that provides the functionality of the reaction pathfinder system 130 or through an application programming interface (API) running on a native operating system of the computing device, such as IOS® or ANDROID™). Some or all of the components of a client device are illustrated in FIG. 7.

The datastore 120 stores reaction data, molecule data, or any suitable data for determining a synthesis route. Reaction data may include a list of reactants in a reaction, a yield or percentage yield, experimental statistics (e.g., a number of experiments performed with a particular reaction), or any suitable data related to a reaction of molecules. Reaction data can include historical reaction data of previously performed reactions. Historical reaction data may be stored in the form of Electronic Laboratory Notebooks (ELNs). The datastore 120 may store historical reaction data that has been processed by a software infrastructure tool such as HazELNut. The historical reaction data may include externally available reactions outside of data from ELNs. Examples of externally available reactions include publicly available reactions or licensable reactions. Molecule data may include formula, mass, structure, or any suitable data related to a molecule. Additional datastores may be included within the environment 100. For example, reaction data may be stored in a first datastore and molecule data may be stored in a second datastore. In some embodiments, the reaction pathfinder system 130 may include a local storage in addition to or alternative to the datastore 120 that stores all or some of the data within the datastore 120.

The reaction pathfinder system 130 uses historical chemical reactions and a reaction network to determine a synthesis route for a user-specified molecule or reaction. The reaction pathfinder system 130 may determine a synthesis route that satisfies one or more criterion to optimize for a synthesis performance metric. The reaction pathfinder system 130 may generate one or more reaction networks using historical chemical reactions (e.g., accessed from the datastore 120). The reaction pathfinder system 130 may synthesize a molecule based on the determined synthesis route. The term “compound” may be used to refer to a molecule, as a compound is a molecule. For example, the term “compound synthesis” may be used to refer to “molecule synthesis.”

The reaction pathfinder system 130 includes software modules such as a network generator 131 and a pathfinder engine 132. The molecule synthesizer 133 may be a combination of hardware and software that includes one or more actuators for initiating a reaction to create a compound and one or more processors for executing software instructions to operate the one or more actuators. The reaction pathfinder system 130 includes or accesses datastores such as the datastore 120. The reaction pathfinder system 130 may have alternative configurations than shown in FIG. 1, including different, fewer, or additional components. For example, the reaction pathfinder system 130 may operate without the molecule synthesizer 133, providing instructions to an external system for synthesizing compounds according to the synthesis route determined by the pathfinder engine 132.

The network generator 131 creates a reaction network representative of reactions among reactants to produce molecules. The network generator 131 may receive historical reaction data to generate the reaction network. The historical reaction data may include ELNs. The historical reaction data may be extracted and processed by a software infrastructure tool that can export or process reaction databases (e.g., HazELNut). The historical reaction data may include externally available reactions outside of data from ELNs. Examples of externally available reactions include publicly available reactions or licensable reactions. The historical reaction data may include data such as a molecule structure, reactants used to produce a molecule, a molecule formula, a molecule mass, moles of a molecule, or any suitable information relating to the molecule or reaction(s) producing the molecule.

Reactions, product molecules, and reactant molecules may be represented as nodes in the reaction network. The reaction network may have a tree structure or any suitable structure for representing reactions between molecules. The tree structure has parent and child nodes. Reaction nodes may be a parent node to two or more molecule child nodes. A molecule node can be a parent node to one or more reaction nodes. In a reaction network having a tree structure, a synthesis route may be a subtree of the reaction network, where the subtree represents one or more reactions and corresponding reaction steps (e.g., a combination of reactants to produce a reaction). The network generator 131 may create edges connecting nodes of the reaction network using synthesis relationships between molecules included within historical reaction data. The network generator 131 may import all or some of the historical reaction data into the reaction network (e.g., annotating a reaction or molecule node with relevant information received in the historical reaction data).

The reaction network may include multiple synthesis routes, and the network generator 131 may determine a synthesis route among the multiple synthesis routes based on a cost of nodes in the synthesis routes. The network generator 131 can assign costs to the nodes in the reaction network. In some embodiments, the network generator 131 assigns an initial cost to molecule nodes based on a source of the molecule. Molecule nodes representing molecules that are readily available (e.g., sourced from a third-party manufacture or any suitable source, such as produced by the reaction pathfinder system 130 itself, that has the molecule available for use in a reaction) or do not otherwise need to be synthesized by the molecule synthesizer 133 may be assigned an initial cost that is lower than an initial cost assigned to a molecule node representing a molecule that is not readily available. For example, the network generator 131 can assign a cost of zero to readily available molecules' nodes and a nonzero cost to nodes representing molecules that are not readily available. The historical reaction data may include information indicating the source of a molecule (e.g., manufacturers that produce the molecule and hence, cause the molecule to be readily available). The cost of a node may depend on the cost of its child nodes. The network generator 131 may iteratively assign costs to nodes as the network generator 131 assigns values to the nodes' child nodes. Example algorithms of assigning costs to nodes are described below.

In some embodiments, the network generator 131 may create a reaction network that optimizes for a criterion that is associated with molecule synthesis performance, which may be referred to as synthesis performance criterion. Examples of synthesis performance criteria for a synthesis route (e.g., relative to other synthesis routes in the reaction network) include having a minimal number of reaction steps, a highest molecule yield, a maximum synthesis robustness, a maximum scale of reaction, a minimal manufacture cost, a minimal number of regulated compounds, or any suitable criterion for evaluating the performance of a route of reactions for synthesizing a molecule. The network generator 131 may assign a cost to a node based on a synthesis performance metric. The network generator 131 may maintain different reaction networks for different synthesis performance criterion.

In a first example of determining the cost of nodes in a reaction network, the network generator 131 determines costs to optimize for the synthesis performance criterion of a minimal number of reaction steps. The network generator 131 may assign an initial cost of zero to nodes representing readily available molecules. For other molecule nodes, the network generator 131 may determine the cost of a molecule node, c(m) where m refers to the molecule, based on the cost of reaction nodes preceding the molecule node. The term “preceding” may refer to nodes that are children node of a particular node. For example, reaction nodes preceding a molecule node, where pred(m) denotes nodes preceding a molecule m, are child nodes of the molecule node that represent reactions that can occur to produce the molecule. The network generator 131 may determine the cost of a reaction node, c(r) where r refers to the reaction, based on a cost of molecule nodes preceding the reaction node. Equations (1) and (2) show example algorithms for calculating c(m) and c(r) for optimizing a minimal number of reactions steps.

$$c ( m ) = {min}_{r ∈ pred ( m )} c ( r ) \qquad ( 1 )$$

$$c ( r ) = 1 + {\sum }_{m ∈ pred ( r )} c ( m ) \qquad ( 2 )$$

In a second example of determining the cost of nodes in a reaction network, the network generator 131 determines costs to optimize for the synthesis performance criterion of a highest total yield. The network generator 131 may assign an initial cost of one to nodes representing readily available molecules. For other molecule nodes, the network generator 131 may determine the cost of a molecule node based on the cost of the reaction node for each reaction child node preceding the molecule node and yield indicators associated with each reaction child node. The yield indicators may be a conversion factor for the product m in reaction r with respect to a limiting reactant and denoted as yield (m, r). The network generator 131 may access values for yield indicators from the historical reaction data. The network generator 131 may determine the cost of a reaction node for a reaction based on a cost of a molecule node corresponding to a molecule that is a limiting reactant for the reaction. Equations (3) and (4) show example algorithms for calculating c(m) and c(r) for optimizing a highest total yield.

$$c ( m ) = {max}_{r ∈ pred ( m )} yield ( m , r ) c ( r ) \qquad ( 3 )$$

$$c ( r ) = c ( m ) \qquad ( 4 )$$

where m=limit(r) in Equation 4, and where limit(r) is the limiting reactant for the reaction r.

In a third example of determining the cost of nodes in a reaction network, the network generator 131 determines costs to optimize for the synthesis performance criterion of a maximum robustness. The network generator 131 may assign an initial cost of infinity or any suitably large value to nodes representing readily available molecules. For example, the initial cost may be a maximum number of experiments recorded as using a particular molecule. For other molecule nodes, the network generator 131 may determine the cost of a molecule node representing a molecule based on a maximum cost of a reaction node for reactions preceding the molecule. The network generator 131 may determine the cost of a reaction node of a reaction based on a number of experiments of the reaction and a minimum molecule node cost of molecule child nodes of the reaction node. Equations (5) and (6) show example algorithms for calculating c(m) and c(r) for optimizing a maximum robustness.

$$c ( m ) = {max}_{r ∈ pred ( m )} c ( r ) \qquad ( 5 )$$

$$c ( r ) = min ( n exp ( r ) , {min}_{m ∈ pred ( r )} c ( m ) ) \qquad ( 6 )$$

where nexp(r) is a measure of the robustness of the reaction r. Examples of measures for robustness of a particular reaction include a number of experiments of the particular reaction, a number of experiments with nonzero yield, or any suitable measure for determining a reliability of the particular reaction.

Additional examples of calculating node costs for synthesis performance metrics may be derived from the initially assigned costs, the Equations 1-6 above, or a combination thereof. For example, the network generator 131 may modify Equations 5 and 6 to determine molecule and reaction node costs to optimize for the synthesis performance metric of maximum scale. In particular, the network generator 131 may replace nexp(r) by a scale of reaction (e.g., expressed in milligrams). In an example for optimizing for the synthesis performance criterion of manufacture costs, the network generator 131 may use Equations 1 and 2 to determine molecule and reaction node costs, but replace the initial cost for readily available molecule nodes with the cost of purchase for the molecule. Additionally, in this example, the network generator 131 may replace the addition of “1” in Equation 2 with the cost of reaction execution (e.g., based on required time of laboratory personnel, amortization or equipment, etc.). The network generator 131 may access the cost of purchase and cost of reaction execution from the historical reaction data. In an example for optimizing for the synthesis performance criterion of regulated compounds, the network generator 131 may modify the algorithm used for optimizing for manufacture cost by including a penalization of regulated intermediates (e.g., toxic compounds). In particular, the network generator 131 may modify the Equation 2 to include the cost of additional procedures required to process regulated intermediates as well as higher costs for their disposal or associated risks. This information for penalizing the use of regulated intermediates may also be received by the network generator 131 in the historical reaction data.

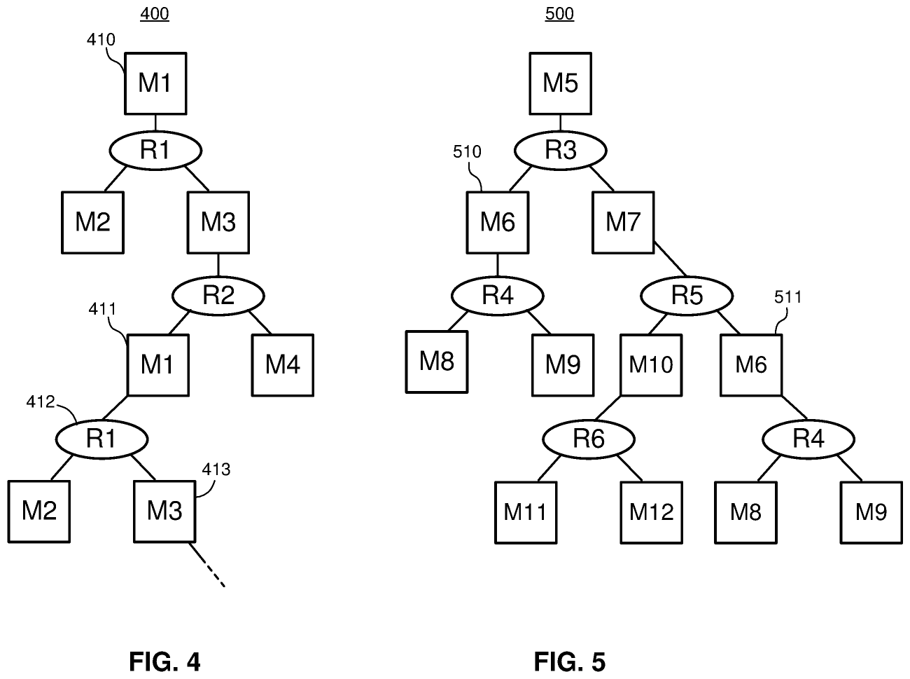

In some embodiments, reactions among molecules may overlap or introduce reaction cycles. Examples of a reaction cycle and a reaction overlap are shown in FIGS. 4 and 5, respectively. A reaction cycle is a reaction to produce a molecule whose reactants include the molecule itself. Cyclic reaction transformations can represent protection and deprotection of highly reactive groups or more complex cyclic transformations present in biochemistry. A reaction overlap includes a molecule that is present at two different locations in a synthesis route. When a reaction overlap is present, the network generator 131 may determine the presence of the reaction overlap and in response, determine a synthesis performance criterion satisfied by the synthesis route.

The network generator 131 may determine node costs for reaction networks including reaction cycles such that the automated generation of the reaction networks are not indefinitely looping with an unknown or unassigned molecule cost. The network generator 131 is capable of bypassing reaction cycles to continue determining costs of nodes in a reaction network. In some embodiments, the network generator 131 identifies a reaction cycle of a molecule node by iterating through predecessor nodes to identify the same molecule node as a child node that has an unassigned cost. As referred to herein, a “predecessor node” may refer to a node that preceded or brought into existence another node (e.g., a reactant node can have molecule nodes as predecessors). Furthermore, a “predecessor node” may be a node indirectly preceding another node, and may not be limited to an immediately preceding node. For example, a predecessor node of a first molecule node may be a reactant used to form a second molecule which is then used to form the first molecule. The network generator 131 then determines cost of the molecule node by ignoring predecessor nodes having unassigned costs. In some embodiments, the network generator 131 determines that a reaction cycle includes a molecule node (e.g., the molecule node that is causing the reaction cycle) that is a reactant of a reaction that is not present in any reaction cycle. The network generator 131 may then assign a predetermined cost to this molecule node. The predetermined cost may be the value of the initial cost (e.g., zero for a reaction network optimizing for minimal reaction steps). Optionally, the network generator 131 can annotate the node with a flag indicating the cost is an approximation. In some embodiments, the network generator 131 may use a similar approximation technique, but the network generator 131 may determine a reaction node that is within a cycle and has at least one of its products outside of any cycles. The network generator 131 may then assign initial costs to these products.

The network generator 131 may update a generated reaction network. The network generator 131 may update a reaction network in response to receiving a request to add a new molecule or reaction to the network. Additionally or alternatively, the network generator 131 may update a reaction network in response to receiving a request to modify data associated with a molecule in the reaction network (e.g., yield of the molecule) or a reaction in the reaction network (e.g., the cost of reaction execution). The network generator 131 may update a generated reaction network automatically and periodically (e.g., weekly, monthly, etc.). In one embodiment of updating a reaction network with an additional molecule, the network generator 131 assigns initial costs to molecules that are not products (e.g., readily available molecules), and iteratively updates costs of the molecule and reaction nodes connected to the newly added molecules.

The network generator 131 can save processing resources by limiting what parts of the reaction network to update. In some embodiments, the network generator 131 can limit a portion of a reaction network that is updated to a portion associated with an optimal cost (e.g., a subtree in a tree network having a branch for a molecule or reaction that is determined by the pathfinder engine 132 to optimize for a synthesis performance criterion relative to other branches). The network generator 131 can keep a list of updated nodes, letting S be the set of the nodes which were not updated, and the network generator 131 can mark one or more predecessor nodes as updated. The network generator 131 can determine an updated cost of a molecule or reaction node only on the nodes with the optimal cost (with respect to the criterion) in the set S. In some embodiments, the network generator 131 can update only nonproduct molecules in a reaction network. In some embodiments, The network generator 131 may update costs of all molecules that have newly added molecule child nodes or updated reaction child nodes.

The pathfinder engine 132 receives a user request to find a synthesis route for a particular molecule or reaction. The pathfinder engine 132 receives a user request specifying a molecule or a reaction. The user request can include molecule parameters defining the chemical composition of a molecule specified or a molecule associated with a specified reaction (e.g., the molecule is a reactant in the specified reaction). An example of a user interface for a user to provide inputs to and receive a synthesis route from the pathfinder engine is shown in FIG. 6.

The pathfinder engine 132 may receive a user request that also defines a synthesis performance metric for which the user wishes to optimize. The pathfinder engine 132 may query a reaction network associated with the specified synthesis performance metric. For example, the pathfinder engine may query a reaction network generated by the network generator 131 to optimize for a highest yield in response to the user request specifying a highest yield as a desired synthesis performance metric.

The pathfinder engine 132 may optionally receive a similarity threshold defining a minimum similarity between a molecule or reaction returned as the parent node at the top of the synthesis route returned by the pathfinder engine 132. The pathfinder engine 132 may determine a level of similarity between molecule parameters provided by the user and a molecule at the top of the returned synthesis route or a molecule serving as a reactant of the reaction at the top of the returned synthesis route. The level of similarity may be based on a similarity in elemental composition, structural composition, or any suitable metric for determining a similarity between atoms and connections thereof within a molecule.

The molecule synthesizer 133 synthesizes one or more molecules according to a synthesis route of a reaction network. In some embodiments, a user of the reaction pathfinder system 130 requests a synthesis route from the pathfinder engine 132 for a particular molecule or reaction. The pathfinder engine 132 may determine a synthesis route (e.g., optimized for a particular synthesis performance metric) and provide the synthesis route to the molecule synthesizer 133 to follow reaction steps in the synthesis route. The molecule synthesizer 133 may include one or more machines or actuators for performing the reaction steps. In some embodiments, the molecule synthesizer can generate instructions for synthesizing molecules autonomously or semi-autonomously.

The network 140 may serve to communicatively couple remote the client device 110, the datastore 120, and the reaction pathfinder system 130. For example, the client device 110 and the reaction pathfinder system 130 are configured to communicate via the network 140. In some embodiments, the network 140 includes any combination of local area and/or wide area networks, using wired and/or wireless communication systems. The network 140 may use standard communications technologies and/or protocols. For example, the network 140 includes communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 140 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network may be represented using any suitable format, such as JavaScript Object Notation (JSON), hypertext markup language (HTML), or extensible markup language (XML). In some embodiments, all or some of the communication links of the network 140 may be encrypted using any suitable technique or techniques.

FIG. 2 is a diagram of a system environment 200 in which the reaction pathfinder system 130 operates, according to one embodiment. The system environment 200 includes remote servers 201, 202, and 203, datastores 210 and 220, and the client device 110. The remote servers 201, 202, and 203 may be managed by different cloud computing services. Although shown as three different remote servers, in some embodiments, there may be more or less servers storing data and performing functions shown. A network such as the network 140 may communicatively couple the remote servers 201, 202, and 203, datastores 210 and 220, and client device 110 shown in the system environment 200.

The diagram of system environment 200 additionally illustrates a process for providing reaction data or molecule data to the reaction pathfinder system 130. The datastore 210A may be a first source of reaction or molecule data (e.g., ELNs) that is mirrored at the datastore 210B of the remote server 201. The data stored in the datastore 210A may be processed from a first file format into a second file format (e.g., RDfile—RDF). A software infrastructure tool for chemical reactions such as HazELNut may process a data file 211 from a first format to a data file 212 of a second format. After the data file 211 is processed, the data file 211 may be deleted or otherwise not stored in the datastore 223. In some embodiments, the file format of the data file 212 produces a smaller file than the file format of the data file 211. In this way, reaction or molecule data may be stored in the datastore 223 while conserving storage resources. Both files 211 and 212 may include data used by the reaction pathfinder system 130. Files in the second format, such as the file 212, may be stored in the datastore 223 at the remote server 202.

The datastore 223 may also store data from the datastore 220, which includes reaction or molecule data from a second source (e.g., from parallel synthesis workbenches (PSW)). The data from the datastore 220 may be in a third format different from formats of the data stored in the datastores 210A and 210B. For example, the data in the datastore 220 may be in a data interchange format such as JSON. The data file 221 provided from the datastore 220 may be converted into the second format for storage at the datastore 223. The data provided from the datastores 210 and 220 may be periodically provided (e.g., daily, weekly, etc.) or provided upon a condition being met (e.g., a threshold amount of new data added to each of the databases 210 or 220, or per user request to extract data from the datastores 210 and 220). Similarly, the reaction pathfinder system 130 may extract data from the datastore 223 periodically, receive the data as pushed from the remote server 202, or queried upon demand. The reaction pathfinder system 130 may receive requests from the client device 110 and provide determined synthesis routes to the client device 110 to fulfill the requests.

### Example Reaction Networks

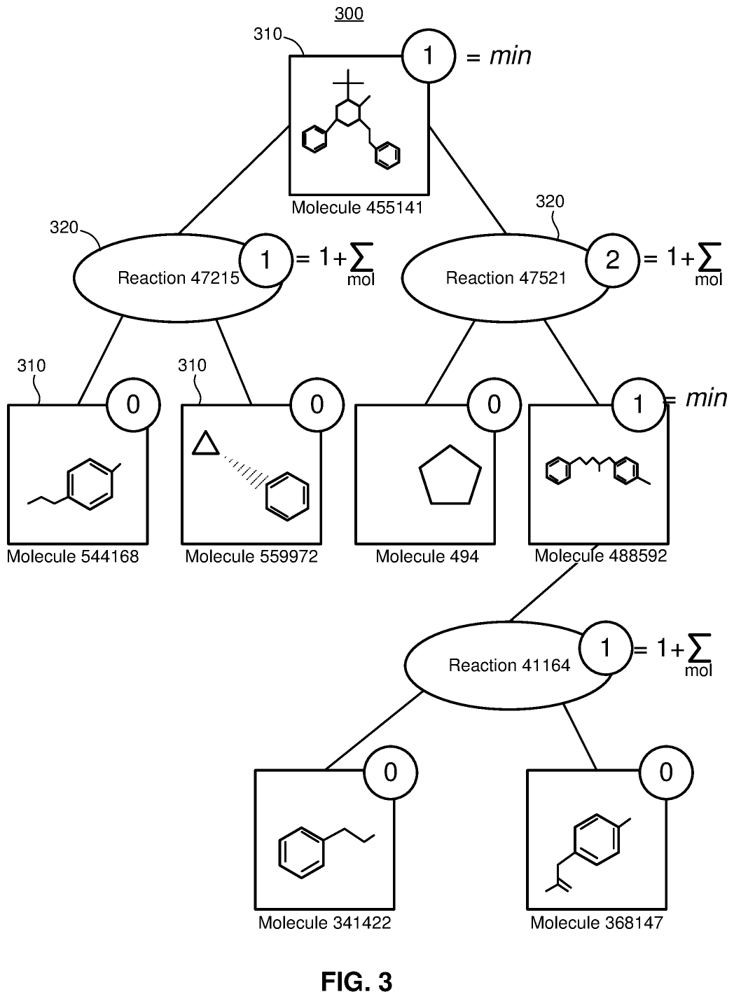

FIG. 3 depicts a structure of a reaction network 300 used by the reaction pathfinder system 130 of FIG. 1, according to one embodiment. The reaction network 300 includes molecule nodes 310 and reaction nodes 320. For clarity, not all reaction nodes and molecule nodes are labeled with reference numerals. The reaction nodes are designated with elliptical shaped nodes and the molecule nodes are designated with square shaped nodes. Each node's cost is designated by circles at the corners of the respective nodes. For example, the cost of the molecule node for molecule ID 455141 is 1.

The reaction network 300 may be generated by the reaction pathfinder system 130. The reaction network 300 is generated to optimize for the synthesis performance metric of a minimal number of reaction steps. In particular, the reaction pathfinder system 130 may use reaction data indicating that the molecule IDs 544168, 559972, 494, 341422, and 368147 are readily available and in response, the reaction pathfinder system 130 may assign an initial cost of zero to the respective molecule nodes. The reaction pathfinder system 130 may then determine reaction node costs based on Equation 2 and subsequently, molecule node costs based on Equation 1. For example, using the initially assigned costs and Equation 2, the reaction pathfinder system may determine the cost of one to assign to both reaction nodes of reaction IDs 47215 and 41164. The reaction pathfinder system 130 may iteratively determine node costs of parent nodes as the costs of child nodes are determined. For example, the reaction pathfinder system 130 may determine the cost of the molecule node associated with molecule ID 488592 using the Equation 1 and reaction child nodes, which is only reaction node for reaction ID 41164 in this embodiment. The equations used to determine certain costs are shown in FIG. 3. For example, the cost for molecule ID 488592 is shown as a minimum of the costs of its predecessor reaction nodes.

As the costs are determined for nodes, the reaction pathfinder system 130 may record which predecessor nodes satisfy the synthesis performance metric for which the reaction network is generated. For example, the reaction pathfinder system 130 (e.g., the network generator 131 or the pathfinder engine 132) may record that the node of reaction ID 47215 satisfies an optimal synthesis route for a minimal number of reaction steps. In embodiments where additional reaction and molecule nodes are included, the reaction pathfinder system 130 may record a chain of reaction or molecule nodes that satisfy a synthesis performance metric.

FIG. 4 depicts a structure of a reaction network 400 including a reaction cycle, according to one embodiment. As shown in the reaction network 400, the molecule M1 contributes to a reaction cycle by appearing as a predecessor node of itself. In particular the molecule M1 appears as at both nodes 410 and 411. The reaction pathfinder system 130 may assign costs to nodes, beginning with assigning values to readily available nodes (e.g., nodes on the reaction network 400 that are not a product of other nodes). The reaction pathfinder system 130 may determine that the reaction network 400 has a reaction cycle. For example, the reaction pathfinder system 130 can identify, using identifiers associated with molecules or corresponding molecule nodes, that the node 411 representing a molecule is a child node of the node 410 representing the same molecule. In response, the reaction pathfinder system 130 may determine that there is a reaction cycle. The reaction pathfinder system 130 may then determine that the subtree at which molecule M1 is at the top (e.g., the subtree with the nodes 410 or 411 at the top) has predecessor molecules with both assigned and unassigned costs. For example, the reaction pathfinder system 130 may determine that the molecule nodes for M2 and M4 have an assigned cost while molecule nodes corresponding to M3 and M1 have unassigned costs. In response, the reaction pathfinder system 130 may determine the cost of molecule nodes for M1 (e.g., using one of the Equations 1, 3, or 5) while ignoring the unassigned predecessor nodes (e.g., M3). In one embodiment, the reaction pathfinder system 130 may ignore a cost of an unassigned predecessor molecule node by approximating the cost as the initial cost for a molecule node. For example, the reaction pathfinder system 130 may calculate the cost of the reaction for reaction R1 at node 412 by setting the cost of the molecule node 413 of molecule M3 to a value of infinity or any suitably large value to optimize for maximum robustness.

In some embodiments, the appearance of the reaction network 400 displayed at a GUI for a user of the reaction pathfinder system 130 to view may be different than what is shown in FIG. 4. In one example, the appearance may omit the appearance of repeated cycles and instead, the reaction network 400 may replace a cyclic node (e.g., the node 411) with an indicator referring to a single appearance of the cyclic node. In one example of an indicator, the reaction pathfinder system 130 may generate an arrow for display that points from reaction R2 to the node 410 that replaces the node 411 and its children from display). In another example of an indicator, the reaction pathfinder system 130 may generate the node 410 for display in a color that distinguishes from other nodes as being a cyclic node.

FIG. 5 depicts a structure of a reaction network 500 including a reaction overlap, according to one embodiment. Molecule M6 at molecule nodes 510 and 511 are reactants that appear twice within a synthesis route (e.g., the subtree with molecule M7 at the top of the subtree). Thus, the molecule M6 causes a reaction overlap in the reaction network 500. The reaction pathfinder system 130 may determine whether there is a reaction overlap present in a reaction network by identifying two or more occurrences of the same molecule within a synthesis route. In some embodiments, the reaction pathfinder system 130 may distinguish a reaction overlap from a reaction cycle by the presence of unassigned node costs indicating a reaction cycle rather than a reaction overlap. In response to identifying a reaction overlap, the reaction pathfinder system 130 may reduce a number of synthesis performance criteria accordingly. For example, molecule M6 present at two different places of a synthesis route can be synthesized only once in a higher amount and used twice, which may result in a lower number of steps.

### Example Pathfinder System User Interface

FIG. 6 shows a graphical user interface (GUI) 600 for using the reaction pathfinder system, according to one embodiment. The GUI 600 includes various user input fields 610, an output field 611, and a reaction network window 612. In some embodiments, the GUI 600 may be generated by the reaction pathfinder system (e.g., the reaction pathfinder system 130). The reaction pathfinder system 130 may generate the GUI 600 for display at a client device (e.g., the client device 110) through a software application or a web browser. The GUI 600 may include fewer, additional, or different components than shown in FIG. 6. For example, the GUI 600 may have additional user input controls for requesting the update of a reaction network with new molecules, reactions, or updated information about an existing molecule or reaction. The GUI 600 may be arranged in a different configuration depending on a type of client device on which it is displayed. For example, in response to determining that the GUI 600 is to be provided to a smartphone, the reaction pathfinder system 130 may arrange the reaction network window 612 to be below the output field 611 rather than side-by-side to conform with a vertically arranged screen of the smartphone.

The user input fields 610 include radio buttons for a user to request a synthesis route for either a reaction ID or molecule ID. The user input fields 610 include a text field for the user to provide molecule parameters describing a molecule for a requested molecule ID, a reactant for a requested reaction ID, or any suitable molecule or reaction synonym (e.g., identification numbers unique to a particular manufacturer or organization). The user input fields 610 include a dropdown menu to specify a similarity threshold percentage that the returned molecule or a reaction's reactant is similar to the provided molecule parameters. The user input fields 610 include a dropdown menu for a synthesis performance criterion, which can include a minimum number of reactions or reaction steps, as shown. The user input fields 610 are examples of user input controls of the GUI 600.

The output field 611 displays some or all of a synthesis route returned in response to the user submitting a request for a molecule ID or a reaction ID. As shown in FIG. 6, the reaction pathfinder system 130 can use a reaction network to determine a synthesis route that satisfies a synthesis performance criterion (e.g., a minimum number of reaction steps) for a molecule having an 80% threshold similarity to molecule parameters specified by the user in the input fields 610. The reaction network window 612 displays all or a portion of a reaction network including the reaction details shown in the output field 611. In some embodiments, the reaction pathfinder system 130 may limit display of a reaction network to a portion (e.g., a subtree) focused on the returned molecule ID or reaction ID (e.g., the corresponding node at the top of the subtree).

### Example Process Using the Pathfinder System

FIG. 7 is a flowchart illustrating a process 700 for determining a synthesis route, according to one embodiment. The reaction pathfinder system 130 may perform the process 700. In some embodiments, the reaction pathfinder system 130 performs operations of the process 700 in parallel or in different orders, or may perform different steps.

The reaction pathfinder system 130 receives 701 a request for a synthesis route. In one example, the pathfinder engine 132 may receive the request. The request may include a user-specified molecule or user-specified reaction. The synthesis route may include a node representing the user-specified molecule or the user-specified reaction. In some embodiments, the user-specified molecule or reaction may be specified by way of molecule parameters describing a molecule corresponding to the user-specified molecule or a reactant of the user-specified reaction. For example, the user may include one or more chemical elements as molecule parameters in the request received 701 by the reaction pathfinder system 130. The request may be provided through a GUI (e.g., the GUI 600 of FIG. 6). The user may submit the request to the reaction pathfinder system 130 in order to determine a synthesis route that optimizes for one or more synthesis performance criteria such as a minimal number of reaction steps.

The reaction pathfinder system 130 queries 702 a reaction network for synthesis routes. In one example, the pathfinder engine 132 may query a reaction network generated by the network generator 131 using historical reaction data. The reaction network may include one or more synthesis routes representing reactions of reactants to produce respective molecules. Each synthesis route may include molecule nodes and reaction nodes. Examples of reaction networks are depicted in FIGS. 3-5. The reaction pathfinder system 130 may query 702 a reaction network based on the molecule parameters provided by the user. In one example, the reaction pathfinder system 130 may compare the information annotating molecule nodes with the user-provided molecule parameters to determine that there is a threshold level of similarity between the chemical elements and structure of a molecule represented in the reaction network and the user-provided molecule parameters. Additionally, the reaction pathfinder system 130 may query 702 a reaction network using a synthesis performance criterion. For example, the reaction pathfinder system 130 may limit the querying 702 to a reaction network having nodes with costs calculated to optimize for the maximum yield of a molecule.

The reaction pathfinder system 130 determines 703, using the reaction network, the synthesis route from the queried synthesis routes. After comparing the molecules or reactions in a reaction network to user inputs provided in the received 701 query, the pathfinder engine 132 may determine 703 a synthesis route in the reaction network that meets a threshold similarity, optimizes for a particular synthesis performance criterion, or a combination thereof. For example, the reaction pathfinder system 130 may identify a molecule node within a reaction network that has at least a threshold similarity with the molecule parameters provided by the user. Each molecule node in the reaction network may be annotated with information regarding the respective molecule, including a composition, structure, or any suitable descriptive information of the molecule that may be mapped to a molecule parameters provided by the user. The reaction pathfinder system 130 may return a portion of the reaction network corresponding to the identified molecule (e.g., a subtree having the molecule as the top node). In some embodiments, the reaction pathfinder system 130 may query a reaction network that has been optimized for a particular synthesis performance criterion and return the portion of that reaction network corresponding to the identified molecule.

### Computing Machine Architecture

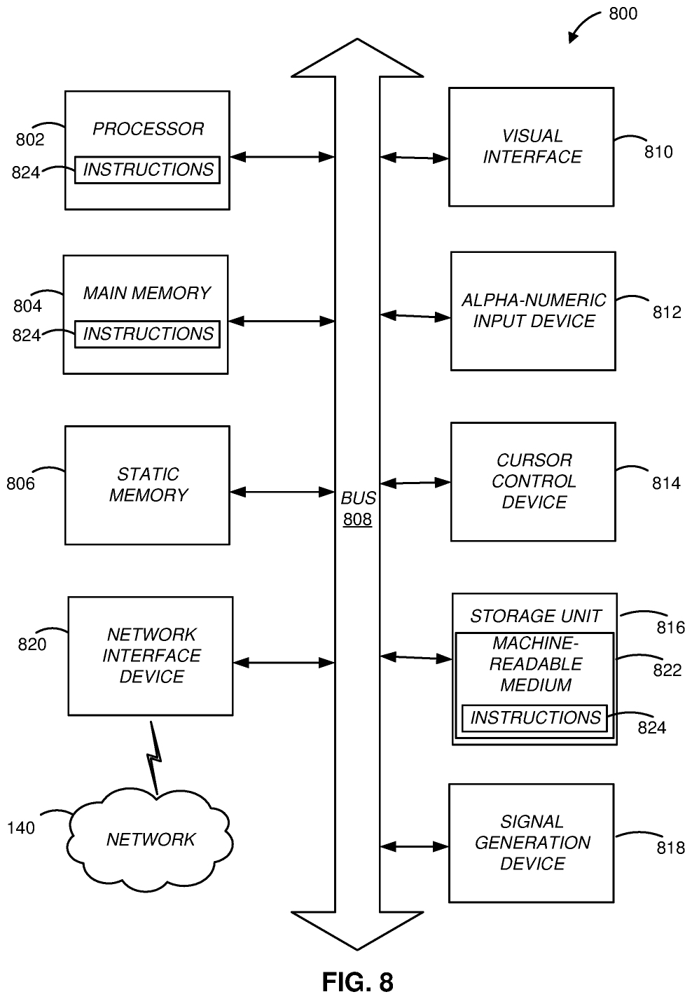

FIG. 8 is a block diagram illustrating components of an example machine able to read instructions from a machine-readable medium and execute them in a processor (or controller). Specifically, FIG. 8 shows a diagrammatic representation of a machine in the example form a computer system 800, within which program code (e.g., software or software modules) for causing the machine to perform any one or more of the methodologies discussed herein may be executed. The program code may be comprised of instructions 824 executable by one or more processors 802. In alternative embodiments, the machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment or connected to a wide area network (WAN) allowing the system's alerts to be sent via email and text messages.

The machine may be a server computer, a client computer, a personal computer (PC), a tablet PC, a set-top box (STB), a personal digital assistant (PDA), a cellular telephone, a smartphone, a web appliance, a network router, switch or bridge, or any machine capable of executing instructions 824 (sequential or otherwise) that specify actions to be taken by that machine. Further, while only a single machine is illustrated, the term “machine” shall also be taken to include any collection of machines that individually or jointly execute instructions 824 to perform any one or more of the methodologies discussed herein.

The example computer system 800 includes a processor 802 (e.g., a central processing unit (CPU), a graphics processing unit (GPU), a digital signal processor (DSP), one or more application specific integrated circuits (ASICs), one or more radio-frequency integrated circuits (RFICs), or any combination of these), a main memory 804, and a static memory 806, which are configured to communicate with each other via a bus 808. The computer system 800 may further include visual display interface 810. The visual interface may include a software driver that enables displaying user interfaces on a screen (or display). The visual interface may display user interfaces directly (e.g., on the screen) or indirectly on a surface, window, or the like (e.g., via a visual projection unit). For ease of discussion the visual interface may be described as a screen. The screen can serve to display a reaction network generated by the reaction pathfinder system or a synthesis route determined by the reaction pathfinder system. The visual interface 810 may include or may interface with a touch enabled screen. The computer system 800 may also include alphanumeric input device 812 (e.g., a keyboard or touch screen keyboard), a cursor control device 814 (e.g., a mouse, a trackball, a joystick, a motion sensor, or other pointing instrument), a storage unit 816, a signal generation device 818 (e.g., a speaker), and a network interface device 820, which also are configured to communicate via the bus 808.

The storage unit 816 includes a machine-readable medium 822 on which is stored instructions 824 (e.g., software) embodying any one or more of the methodologies or functions described herein. The instructions 824 (e.g., software) may also reside, completely or at least partially, within the main memory 804 or within the processor 802 (e.g., within a processor's cache memory) during execution thereof by the computer system 800, the main memory 804 and the processor 802 also constituting machine-readable media. The instructions 824 (e.g., software) may be transmitted or received over a network 270 via the network interface device 820.

While machine-readable medium 822 is shown in an example embodiment to be a single medium, the term “machine-readable medium” should be taken to include a single medium or multiple media (e.g., a centralized or distributed database, or associated caches and servers) able to store instructions (e.g., instructions 824). The term “machine-readable medium” shall also be taken to include any medium that is capable of storing instructions (e.g., instructions 824) for execution by the machine and that cause the machine to perform any one or more of the methodologies disclosed herein. The term “machine-readable medium” includes, but not be limited to, data repositories in the form of solid-state memories, optical media, and magnetic media.

### Additional Considerations

The reaction pathfinder system described herein has numerous benefits and advantages. The reaction pathfinder system enables a graph algorithm and tool to enable users to query past reactions with an increased speed and scale as compared to manually querying historical reaction data (e.g., ELNs). This then saves users a significant amount of time and costs. A search for a synthesis route having a minimal number of reaction steps that takes hours with a manual query may be completed in seconds (e.g., fifteen seconds). The reaction pathfinder system can also export all reaction steps to a word processing document along with a listing of ELN notebook page references. By finding optimal synthesis routes for synthesizing a molecule, the pathfinder reaction system also positively impacts a discovery pipeline for new molecules. By finding an optimal synthesis route that improves a percentage yield of a molecule, the pathfinder reaction system can increase the chemical resources and cost (e.g., labor and time) saved, as the amount of molecules being manufactured is maximized.

Furthermore, the reaction pathfinder system increases the speed of knowledge transfer between laboratories or users as projects in chemistry are frequently stopped and resumed even by different groups. Without the organization of historical reactions and ability to query reaction networks that is provided by the reaction pathfinder system, users can find difficulty in quickly understanding which synthesis routes were tried and which were most successful. The reaction pathfinder system enables users to speed up this process of understanding previous experiments. In this way, the reaction pathfinder system can also serve as a learning tool for junior scientists.

Additionally, the reaction pathfinder system may reduce time expended by a user for resynthesizing packages for external partners. The reaction pathfinder system allows a user to export reaction networks and data obtained from querying reaction networks in minutes rather than hours. Also, finding late-stage intermediates that are internally or externally available may save a considerable number of reactions performed in the laboratory (e.g., some synthesis routes can have more than twenty steps). Finally, the reaction pathfinder system enables a quick assessment of the robustness of chemistry on synthesis routes, which allows users to choose the most promising synthesis routes which also saves performed reactions and associated time.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The performance of certain of the operations may be distributed among the one or more processors, not only residing within a single machine, but deployed across a number of machines. It should be noted that where an operation is described as performed by “a processor,” this should be construed to also include the process being performed by more than one processor. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. The one or more processors may also operate to support performance of the relevant operations in a “cloud computing” environment or as a “software as a service” (SaaS).

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise. As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Upon reading this disclosure, those of skill in the art will appreciate still additional alternative structural and functional designs for a system and a process for automated quality check and diagnosis for production model refresh through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A non-transitory computer readable medium comprising stored instructions that, when executed by a computing device, cause the computing device to:
receive a request for a synthesis route comprising a user-specified molecule or a user-specified reaction;
query a reaction network comprising possible synthesis routes, the reaction network comprising molecule nodes and reaction nodes, the reaction network representative of reactions of reactants to produce respective molecules; and
determine, using the reaction network, the synthesis route from the possible synthesis routes.

2. The non-transitory computer readable medium of claim 1, wherein the determined synthesis route optimizes for one or more synthesis performance criteria including a minimum number of reaction steps for producing a molecule, a maximum yield of the molecule, or a maximum robustness for producing the molecule.

3. The non-transitory computer readable medium of claim 1, wherein the instructions further cause the computing device to:
identify a reaction cycle within the reaction network, wherein the reaction cycle is a portion of the reaction network caused by a molecule child node for a molecule serving as a predecessor node of a molecule parent node for the molecule;
identify a molecule node within the reaction cycle having an unassigned cost; and
assign the molecule node within the reaction cycle a predetermined cost.

4. The non-transitory computer readable medium of claim 1, wherein the instructions further cause the computing device to generate the reaction network using historical reaction data.

5. The non-transitory computer readable medium of claim 4, wherein the instructions further cause the computing device to assign initial cost values to the reaction network based on a plurality of sources of the molecules represented in the reaction network.

6. The non-transitory computer readable medium of claim 5, wherein the instructions further cause the computing device to:
assign a first value for a first node of the molecule nodes representative of a molecule sourced from a third-party; and
assign a second value for a second node of the molecule nodes representative of a molecule produced using the molecule sourced from the third-party, the first value smaller than the second value.

7. The non-transitory computer readable medium of claim 4, wherein the instructions further cause the computing device to determine a reaction node cost for a reaction node connected to molecule child nodes having assigned costs.

8. The non-transitory computer readable medium of claim 7, wherein the instructions further cause the computing device to:
in response to determining to optimize against a synthesis performance criterion of a minimum number of reaction steps for producing a molecule:
assign an initial cost of zero to a subset of the molecule nodes;
determine the reaction node cost based on costs of molecule child nodes of the reaction node; and
determine a molecule node cost of a molecule node based on a minimum of costs of reaction child nodes of the molecule node.

9. The non-transitory computer readable medium of claim 7, wherein the instructions further cause the computing device to:
in response to determining to optimize against a synthesis performance criterion of a maximum yield of a molecule:
assign an initial cost of one to a subset of the molecule nodes;
determine the reaction node cost based on a cost of a molecule node corresponding to a limiting reactant associated with the reaction node; and
determine a molecule node cost based on yield indicators and a cost of child nodes of the molecule node.

10. The non-transitory computer readable medium of claim 7, wherein the instructions further cause the computing device to:
in response to determining to optimize against a synthesis performance criterion of a maximum robustness for producing a molecule:
assign an initial cost to a subset of the molecule nodes, wherein the initial cost is larger than a maximum number of experiments using a particular molecule of the synthesis routes in the reaction network;
determine the reaction node cost based on a number of experiments of a reaction represented by the reaction node and based on a minimum cost of molecule child nodes of the reaction node; and
determine a molecule node cost based on a maximum cost of reaction child nodes of the molecule node.

11. The non-transitory computer readable medium of claim 1, wherein the instructions further cause the computing device to synthesize a molecule based on the determined synthesis route.

12. The non-transitory computer readable medium of claim 1, wherein the instructions further cause the computing device to generate a graphical user interface (GUI) comprising one or more user input controls for receiving the request.

13. The non-transitory computer readable medium of claim 12, wherein the instructions further cause the computing device to generate the GUI comprising a user input control for specifying a frequency at which the reaction network is to be updated.

14. The non-transitory computer readable medium of claim 1, wherein the instructions further cause the computing device to:
identify a portion of the reaction network based on a presence of a particular synthesis route within the portion of the reaction network, the particular synthesis route optimizing for a synthesis performance criterion; and
update the portion of the reaction network to include additional molecule data or additional reaction data.

15. A system for determining a synthesis route within a reaction network, the system comprising:
a datastore that stores the reaction network, the reaction network comprising a plurality of molecule nodes and a plurality of reaction nodes, the reaction network representative of reactions of reactants to produce respective molecules; and
a pathfinder engine configured to:
receive a request for the synthesis route comprising one of a user-specified molecule or a user-specified reaction;
query the reaction network comprising a plurality of synthesis routes; and
determine, using the reaction network, the synthesis route from the plurality of synthesis routes.

16. The system of claim 15, further comprising:
a network generator configured to:
generate the reaction network using historical reaction data;
assign initial cost values to the reaction network based on a plurality of sources of molecules represented in the reaction network; and
determine a reaction node cost for a reaction node connected to molecule child nodes having assigned costs.

17. The system of claim 15, further comprising:
a molecule synthesizer configured to synthesize a molecule based on the determined synthesis route.

18. A method for determining a synthesis route within a reaction network, the method comprising:
receiving a request for the synthesis route comprising one of a user-specified molecule or a user-specified reaction;
querying the reaction network comprising a plurality of synthesis routes, the reaction network comprising a plurality of molecule nodes and a plurality of reaction nodes, the reaction network representative of reactions of reactants to produce respective molecules; and
determining, using the reaction network, the synthesis route from the plurality of synthesis routes

19. The method of claim 18, further comprising:
generating the reaction network using historical reaction data;
assigning initial cost values to the reaction network based on a plurality of sources of molecules represented in the reaction network; and
determining a reaction node cost for a reaction node connected to molecule child nodes having assigned costs.

20. The method of claim 18, further comprising synthesizing a molecule based on the determined synthesis route.
